(19) United States
(12) Patent Application Publication
(10) Pub. No.: US 2018/0178262 A1
(43) Pub. Date: Jun. 28, 2018

(54) METHOD FOR CARRYING OUT ANAEROBIC DIGESTION BY USING HEAVY METAL IONS-RICH BIOMASS WASTE MATERIALS

(71) Applicant: Peking University Shenzhen Graduate School, Shenzhen (CN)

(72) Inventors: Qiyong XU, SHENZHEN (CN); Jae Hac KO, SHENZHEN (CN); Ning WANG, SHENZHEN (CN); Yuqing LIAO, SHENZHEN (CN); Songwei BIAN, SHENZHEN (CN)

(73) Assignee: Peking University Shenzhen Graduate School

(21) Appl. No.: 15/853,728

(22) Filed: Dec. 23, 2017

(30) Foreign Application Priority Data
Dec. 26, 2016 (CN) 201611216343.7

(51) Int. Cl. B09B 3/00; C12M 1/107

(52) U.S. Cl. CPC B09B 3/0083; C12M 21/04

(57) ABSTRACT

Provided is a method for performing anaerobic digestion by utilizing a heavy metal ion enriched biomass waste material, comprising: putting a biochar made from the raw materials such as garden rubbish and agricultural and forestry waste, or a plant for adsorbing heavy metal ions in waste water containing heavy metal ions, and obtaining a heavy metal saturated biochar or a heavy metal super-enriched plant; and then adding in a kitchen waste anaerobic digestion methane generation system at a certain ratio. The heavy metal ions adsorbed in the plant or the biochar are utilized as a basic component of an anaerobic reaction enzyme to promote the growth of methane bacteria, activate the activity of the enzyme, shorten the time of a hydrolysis acidification stage, reduce VFA accumulation, and accelerate the generation of energy products such as methane and the like.

### CROSS-REFERENCE TO RELATED APPLICATIONS

This application claims priority to Chinese Patent Application No. 201611216343.7 with a filing date of Dec. 26, 2016. The content of the aforementioned applications, including any intervening amendments thereto, are incorporated herein by reference.

### TECHNICAL FIELD

The present invention relates to the technical field of environmental protection and the field of new energy development and utilization, in particular to a method for performing anaerobic digestion by utilizing a heavy metal ion enriched biomass waste material.

### BACKGROUND

In recent years, heavy metal waste water increases with the development of the industry. Domestic waste water and industrial waste water contain the metals of Cu, Zn, Co, Ni and the like. If the waste water is not treated properly, the heavy metal will generate huge hazard to natural environment and human health. The common heavy metal waste water treatment methods at present comprise a chemical precipitation method, oxidation reduction method, ion exchange method and the like. However, existing methods unavoidably have the problems of hard reclamation, small treatment amount, and high cost. Therefore, developing a low cost and high efficiency treatment method is extremely important. Biochar adsorption method or plant adsorption method for adsorbing heavy metal ions refers to the method for adsorbing the heavy metal in waste water by utilizing biochar or a plant having a heavy meal enrichment effect. However, at present, the methods for reclaiming the biochar or the super-enriched plant having adsorbed heavy metals are few. The proposed treatment methods comprise an incineration method, a compression landfilling method, a composting method and the like. However, the technologies are mostly characterized in that the biochar or the super-enriched plant having adsorbed heavy metals are treated as rubbish or hazard waste material, instead of being comprehensively utilized. And the environment will be polluted when the rubbish is treated with any one of the above-described methods: the hazard waste is not cleaned thoroughly; the final product is still risky, and is easy to generate “secondary pollution”. Under the biomagnification effect of the food chain, the heavy metals are enriched in thousands of times, have potential toxicity, and finally endanger human and other organisms through the food chain. Therefore, using biochar or plant to adsorb metal only transfers the metal from one place to another place, but cannot fundamentally solve the heavy metal pollution problem.

### SUMMARY OF THE INVENTION

The technical problems to be solved by the present invention are providing a method for performing anaerobic digestion by utilizing a heavy metal ion enriched biomass waste material, and realizing the reduction, reclamation and utilization of both kitchen waste and the heavy metal ion enriched biomass waste material.

To solve the above-described technical problem, the present invention provides a method for performing anaerobic digestion by utilizing a heavy metal ion enriched biomass waste material, comprising:

Step S1, obtaining a heavy metal ion enriched biomass waste material;

Step S2, determining a mixing ratio of the heavy metal ion enriched biomass waste material and kitchen waste according to a heavy metal concentration range having a promotion effect on anaerobic digestion and the concentration of the heavy metal ions in the heavy metal ion enriched biomass waste material;

Step S3, adding the heavy metal ion enriched biomass waste material in a kitchen waste anaerobic digestion reactor according to the mixing ratio, and uniformly mixing with the kitchen waste;

Step S4, performing upper layer intermittent aeration pretreatment on the mixed kitchen waste and heavy metal ion enriched biomass waste material;

Step S5, after the pH value in the anaerobic digestion reactor raises above 7.0, stopping aeration, and recirculating a leachate;

Step S6, collecting gases generated in the anaerobic digestion reactor, periodically monitoring the contents of methane and hydrogen in the generated gases, and calculating a daily methane yield and a cumulative methane yield; and

Step S7, sampling the reactants in the anaerobic digestion reactor, measuring the concentration of the heavy metal ions therein, and calculating the utilization ratio of the heavy metal ions.

Wherein the step S1 specifically comprises:

Thermal cracking garden rubbish and agricultural and forestry waste into a biochar under an anoxic condition; and

Putting the biochar in waste water containing heavy metal ions, and obtaining a heavy metal saturated biochar.

Wherein the step S1 specifically comprises:

Putting a plant for adsorbing heavy metal ions in the waste water containing heavy metal ions, and obtaining a heavy metal super-enriched plant.

Wherein the plant for adsorbing heavy metal ions comprises water hyacinth, ciliate desert-grass, penny cress, Indian cabbage mustard, and sedum alfredii hance.

Wherein in the step S3, before adding the kitchen waste and the heavy metal ion enriched biomass waste material in the kitchen waste anaerobic digestion reactor, further comprising:

Crushing the kitchen waste with the particle diameter controlled less than 5 mm.

Wherein in the heavy metal ion enriched biomass waste material added in the anaerobic digestion reactor, the content of Ni is 50-300 mg/kg total solid (TS); the content of Co is 2-6 mg/kg TS; the content of Fe is 1000-5000 mg/kg TS; the content of Zn is 200-600 mg/kg TS; the content of Mn is 200-500 mg/kg TS; the content of Mo is 3-30 mg/kg TS, wherein the total solid (TS) means the content of the remaining solids after the moisture is removed from an anaerobic digestion substrate.

Wherein if the measured concentration of the heavy metal ions in the sampled reactants is low in the step S7, then adding the plant for adsorbing heavy metal ions put in the waste water containing heavy metal ions.

Wherein the method further comprises:

In the late stage of methanation, the stable rubbish having been treated by the heavy metal ion enriched biomass waste material is landfilled at a final landfill; and the leachate is delivered to a urban domestic sewage treatment plant for deep treatment before being discharged.

The present invention has the following beneficial effects:

On the basis of existing rubbish reclamation and utilization, the heavy metal saturated biochar or the heavy metal super-enriched plant are added in the kitchen waste anaerobic digestion methane generation system at a certain ratio; the heavy metal ions adsorbed in the plant or the biochar are utilized as a basic component of an anaerobic reaction enzyme to promote the growth of methane bacteria, activate the activity of the enzyme, improve the organic load of the anaerobic digestion, shorten the time of a hydrolysis acidification stage, reduce VFA accumulation, and accelerate the generation of energy products such as methane and the like. Therefore, the present invention not only can treat the biochar o the super-enriched plant waste material, eliminate the secondary pollution of the heavy metal saturated biochar or the heavy metal super-enriched plant to the environment, reduce rubbish, realize harmless rubbish utilization, but also can promote anaerobic digestion methane generation, thus having an important significance for accelerating the treatment and reclamation of kitchen waste.

### BRIEF DESCRIPTION OF THE DRAWINGS

In order to illustrate the technical schemes in the embodiments of the present invention or in the prior art more clearly, the drawings which are required to be used in the description of the embodiments or the prior art are briefly described below. It is obvious that the drawings described below are only some embodiments of the present invention. It is apparent to those of ordinary skill in the art that other drawings may be obtained based on the accompanying drawings without inventive effort.

FIG. 1 is a flow chart of the method for performing anaerobic digestion by utilizing a heavy metal ion enriched biomass waste material according to the embodiment of the present invention; and

FIG. 2 is a schematic diagram of the embodiment of the present invention.

### DETAILED DESCRIPTION OF THE EMBODIMENTS

The description of the embodiments hereafter is used to illuminate a specific embodiment the present invention can implement with reference to figures.

On the basis of an anaerobic digestion reactor, the present invention develops a novel and efficient urban kitchen waste landfilling treatment technology, and utilizes a heavy metal ion enriched biomass waste material to solve the existing problems of too long hydrolysis and acidification time, slow rubbish degradation speed, poor methane generation effect, VFA accumulation and the like in the anaerobic digestion process of the kitchen waste.

Anaerobic digestion methane generation is divided into four stages: a hydrolysis stage, an acidification stage, an acetic acid generation stage, and a methane generation stage. In the early stage, the substrate is hydrolyzed and acidified to generate a great deal of volatile fatty acid (VFA) and soluble chemical oxygen demand (SCOD); the substrate having a high protein content is hydrolyzed to generate a great deal of VFA, wherein the VFA reduces the pH value of the whole system, which is adverse to the growth of methane bacteria, and results in methane generation failure. Adding a microelement (e.g. Fe, Co, Ni, Se) in the reactor not only can promote the growth of the methane bacteria, activate the activity of the enzyme, improve the organic load capability of the anaerobic digestion, stabilize the reaction, but also can improve the yield and generation rate of the methane. For example, adding Fe in the anaerobic digestion reactor can increase soluble Fe, reduce the concentrations of the VFA and the SCOD in the anaerobic digestion, and transform the reaction conditions to the direction facilitating methane generation; and adding the microelement Co can also reduce the concentrations of the VFA and the SCOD. Therefore, the present invention re-utilizes the heavy metal ion adsorbed biochar or the super-enriched plant by putting in the anaerobic digestion reaction, and promotes the anaerobic digestion methane generation process.

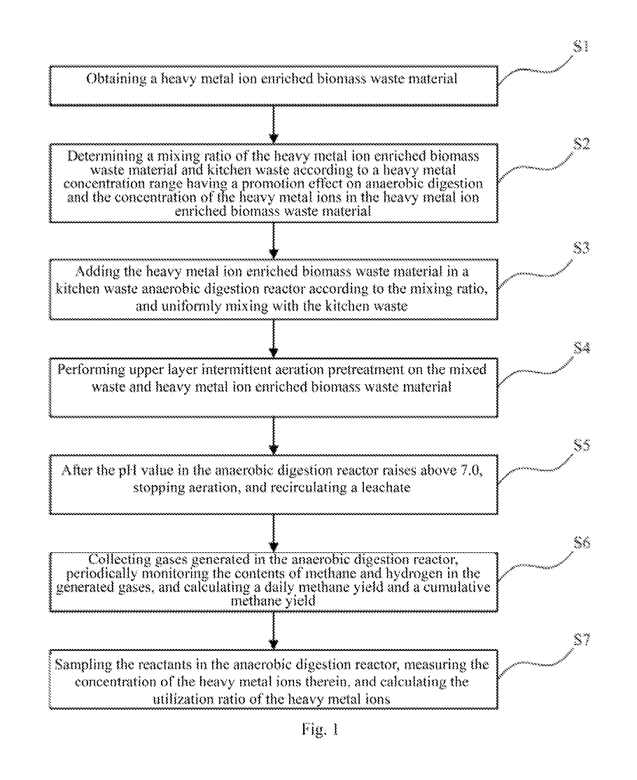

With reference to FIG. 1, the embodiment of the present invention provides a method for performing anaerobic digestion by utilizing a heavy metal ion enriched biomass waste material, comprising:

Step S1, obtaining a heavy metal ion enriched biomass waste material;

Step S2, determining a mixing ratio of the heavy metal ion enriched biomass waste material and kitchen waste according to a heavy metal concentration range having a promotion effect on anaerobic digestion and the concentration of the heavy metal ions in the heavy metal ion enriched biomass waste material;

Step S3, adding the heavy metal ion enriched biomass waste material in a kitchen waste anaerobic digestion reactor according to the mixing ratio, and uniformly mixing with the kitchen waste;

Step S4, performing upper layer intermittent aeration pretreatment on the mixed kitchen waste and heavy metal ion enriched biomass waste material;

Step S5, after the pH value in the anaerobic digestion reactor raises above 7.0, stopping aeration, and recirculating a leachate;

Step S6, collecting gases generated in the anaerobic digestion reactor, periodically monitoring the contents of methane and hydrogen in the generated gases, and calculating a daily methane yield and a cumulative methane yield; and

Step S7, sampling the reactants in the anaerobic digestion reactor, measuring the concentration of the heavy metal ions therein, and calculating the utilization ratio of the heavy metal ions.

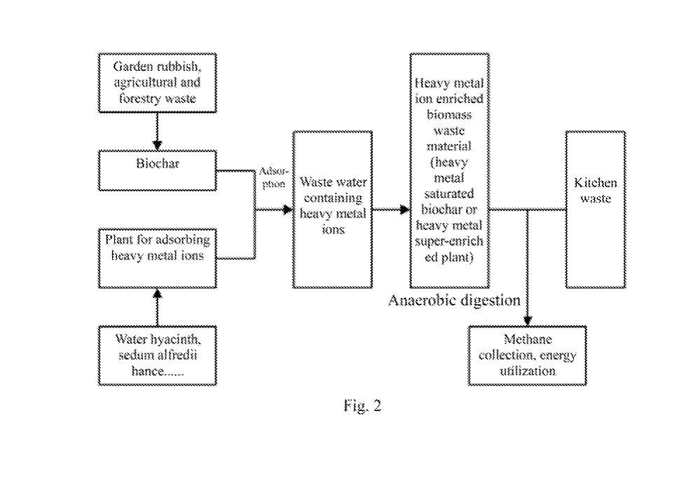

The embodiment will be elaborated hereafter in connection with FIG. 2.

In step S1, the heavy metal ion enriched biomass waste material mainly has two sources:

(1) the biochar, a stable carbon rich product made by thermal cracking a biomass waste material such as urban garden organic rubbish, agricultural and forestry waste and the like under an anoxic condition. The biochar is added in the waste water containing heavy metal ions; the metal ions and functional groups on the surface of the biochar form a specific metal complex, such that the metal ions can be stabilized in the water, namely obtaining a heavy metal. The biochar has a large specific surface area and great surface energy, has a strong heavy metal ion combination propensity, and further has a good adsorption effects for a plurality of heavy metals such as nickel, lead, copper, zinc and the like. After the heavy metal ions are saturated, the heavy metal saturated biochar loses the original heavy metal adsorption capability, and requires to be treated. In the embodiment, the heavy metal saturated biochar is put in the kitchen waste anaerobic digestion reactor to improve the methane generation efficiency.

As can be seen from above, in the embodiment, the biochar is made from waste biomass material, which equivalents to primary waste utilization; then the biochar is put in the waste water containing heavy metal ions to adsorb heavy metal ions, thus having the effect of sewage treatment; and finally when the obtained heavy metal saturated biochar requires to be treated, the heavy metal saturated biochar is added in the kitchen waste anaerobic digestion reactor, which equivalents to secondary waste utilization.

(2) Plant for adsorbing heavy metal ions A few aquatic herbaceous plants, such as water hyacinth, have a good purification effect for the heavy metal ions in sewage, and can remove the heavy metal ions in the environment or reduce the concentration thereof through the actions of rhizofiltration, volatilization, absorption, enrichment and the like. A few other plants, such as ciliate desert-grass, penny cress, Indian cabbage mustard, sedum alfredii hance and the like, can adsorb, deliver and accumulate high concentration of heavy metals in the soil. However, after the plant adsorbs the heavy metals and becomes a heavy metal super-enriched plant, the plant is required to be treated properly, and cannot be used as fodders due to the heavy metals contained therein. In the embodiment, the heavy metal super-enriched plant is also put in the kitchen waste anaerobic digestion reactor to improve the methane generation efficiency.

Likewise, the embodiment utilizes the plant having the heavy metal adsorption capability to adsorb the heavy metal ions in the waste water containing heavy metal ions, thus having the effect of sewage treatment; after the heavy metal ions are adsorbed, the plant becomes a heavy metal super-enriched plant, and is required to be treated. Then the heavy metal super-enriched plant is put in the kitchen waste anaerobic digestion reactor, which equivalents to waste utilization.

In step S2, different anaerobic digestion substrates contain different ingredients, for example, the contents of fat, starch, cellulose and metal are different. Therefore, the bacteria for hydrolysis are also different; and different bacteria have different microelements demands. Hence, the concentration of the heavy metal having a promotion effect on the anaerobic digestion is a range. In addition, the mixing ratio of the heavy metal ion enriched biomass waste material and the kitchen waste is used to ensure that adequate heavy metal ions are provided to promote the anaerobic digestion during anaerobic digestion; therefore, the specific range of the mixing ratio is required to be determined according to the concentration of the heavy metal ions.

In step S3, before the kitchen waste and the heavy metal ion enriched biomass waste material are delivered to the kitchen waste anaerobic digestion reactor, the kitchen waste is required to be crushed with the particle diameter controlled less than 5 mm, thus facilitating the rapid degradation of substances in subsequent steps.

It should be noted that the heavy metal saturated biochar and the heavy metal super-enriched plant are generally separately mixed with the kitchen waste and then the mixture is put in the kitchen waste anaerobic digestion reactor; namely, the heavy metal saturated biochar is mixed with kitchen waste and then the mixture is put in the kitchen waste anaerobic digestion reactor; or the heavy metal super-enriched plant is mixed with kitchen waste and then the mixture is put in the kitchen waste anaerobic digestion reactor.

In the heavy metal ion enriched biomass waste material added in the anaerobic digestion reactor, the content of Ni is 50-300 mg/kg total solid (TS which means the content of the remaining solids after the moisture is removed from the anaerobic digestion substrate in the embodiment); the content of Co is 2-6 mg/kg TS; the content of Fe is 1000-5000 mg/kg TS; the content of Zn is 200-600 mg/kg TS; the content of Mn is 200-500 mg/kg TS; and the content of Mo is 3-30 mg/kg TS.

It should be noted that the process in the present invention that the heavy metal ion enriched biomass waste material (the heavy metal saturated biochar or the heavy metal super-enriched plant) is added in the kitchen waste anaerobic digestion reactor, adsorbs enriched heavy metal ions, and transfers the heavy metal ions to an anaerobic digestion fermentation system through the growth of a microorganism on the surface of the material, is a slow release process which can remain the constant concentration of the heavy metal ions in the anaerobic digestion system. Therefore, the present invention is more advantageous than directly adding a specific concentration of heavy metal solution.

In step S5, after the pH value in the anaerobic digestion reactor raises above 7.0, stopping aeration, and starting an active stage for generating methane. In this case, the upper rubbish in the reactor is in a methane generation environment, and the lower rubbish is still in the acidification stage. By means of leachate recirculation one time per day, the upper rubbish is replenished by the lower organism, thus improving the methane generation capability thereof; and the lower rubbish is inoculated and diluted by the upper microorganism, thus promoting the establishment of the lower methane generation environment.

It should be noted that the daily methane yield can perfectly reflect the daily methane generation rate of the whole system; and the cumulative methane yield can use the total methane yield generated in the whole reaction stages to directly denote the energy utilization situation. Therefore, the effects of periodically monitoring the contents of methane and hydrogen in the generated gases, and calculating the daily methane yield and the cumulative methane yield in step S6 are: capable of more intuitively and quantitatively reflecting the efficient reclamation degree of the whole system via the two measurement indexes.

The reason why the content concentration of the heavy metal ions is measured and the utilization ratio of the heavy metal ions is calculated in step 7 is that a too high heavy metal ion concentration will inhibit the generation of the methane, thus the concentration of the heavy metal ions is required to be controlled in a certain range. If the required heavy metal ion concentration is low, then the concentration can be improved by adding the heavy metal super-enriched plant. Namely, if the measured concentration of the heavy metal ions in the sampled reactants is low in the step S7, then adding the plant for adsorbing heavy metal ions put in the waste water containing heavy metal ions.

In the late stage of methanation, the stable rubbish having been treated by the heavy metal ion enriched biomass waste material is landfilled at a final landfill; and the leachate is delivered to a urban domestic sewage treatment plant nearby for deep treatment before being discharged.

From the description above, the present invention has the following beneficial effects:

On the basis of existing rubbish reclamation and utilization, the heavy metal saturated biochar or the heavy metal super-enriched plant are added in the kitchen waste anaerobic digestion methane generation system at a certain ratio; the heavy metal ions adsorbed in the plant or the biochar are utilized as a basic component of an anaerobic reaction enzyme to promote the growth of methane bacteria, activate the activity of the enzyme, improve the organic load of the anaerobic digestion, shorten the time of a hydrolysis acidification stage, reduce VFA accumulation, and accelerate the generation of energy products such as methane and the like. Therefore, the present invention not only can treat the biochar o the super-enriched plant waste material, eliminate the secondary pollution of the heavy metal saturated biochar or the heavy metal super-enriched plant to the environment, reduce rubbish, realize harmless rubbish utilization, but also can promote anaerobic digestion methane generation, thus having an important significance for accelerating the treatment and reclamation of kitchen waste.

The disclosure above is only the preferred embodiments of the present invention, but not intended to limit the protection scope of the present invention. Therefore, any equivalent variations made according to the claims of the present invention are all concluded in the protection scope of the present invention.

We claim:

1. A method for performing anaerobic digestion by utilizing a heavy metal ion enriched biomass waste material, comprising:
Step S1, obtaining a heavy metal ion enriched biomass waste material;
Step S2, determining a mixing ratio of the heavy metal ion enriched biomass waste material and kitchen waste according to a heavy metal concentration range having a promotion effect on anaerobic digestion and the concentration of the heavy metal ions in the heavy metal ion enriched biomass waste material;
Step S3, adding the heavy metal ion enriched biomass waste material in a kitchen waste anaerobic digestion reactor according to the mixing ratio, and uniformly mixing with the kitchen waste;
Step S4, performing upper layer intermittent aeration pretreatment on the mixed kitchen waste and heavy metal ion enriched biomass waste material;
Step S5, after the pH value in the anaerobic digestion reactor raises above 7.0, stopping aeration, and recirculating a leachate;
Step S6, collecting gases generated in the anaerobic digestion reactor, periodically monitoring the contents of methane and hydrogen in the generated gases, and calculating a daily methane yield and a cumulative methane yield; and
Step S7, sampling the reactants in the anaerobic digestion reactor, measuring the concentration of the heavy metal ions therein, and calculating the utilization ratio of the heavy metal ions.

2. The method according to claim 1, characterized in that, the step S1 specifically comprises:
Thermal cracking garden rubbish and agricultural and forestry waste into a biochar under an anoxic condition; and
Putting the biochar in waste water containing heavy metal ions, and obtaining a heavy metal saturated biochar.

3. The method according to claim 1, characterized in that, the step S1 specifically comprises:
Putting a plant for adsorbing heavy metal ions in the waste water containing heavy metal ions, and obtaining a heavy metal super-enriched plant.

4. The method according to claim 3, characterized in that, the plant for adsorbing heavy metal ions comprises water hyacinth, ciliate desert-grass, penny cress, Indian cabbage mustard, and sedum alfredii hence.

5. The method according to claim 1, characterized by, in the step S3, before adding the kitchen waste and the heavy metal ion enriched biomass waste material in the kitchen waste anaerobic digestion reactor, further comprising: crushing the kitchen waste with the particle diameter controlled less than 5 mm.

6. The method according to claim 1, characterized in that, in the heavy metal ion enriched biomass waste material added in the anaerobic digestion reactor, the content of Ni is 50-300 mg/kg total solid (TS); the content of Co is 2-6 mg/kg TS; the content of Fe is 1000-5000 mg/kg TS; the content of Zn is 200-600 mg/kg TS; the content of Mn is 200-500 mg/kg TS; the content of Mo is 3-30 mg/kg TS, wherein the total solid (TS) means the content of the remaining solids after the moisture is removed from an anaerobic digestion substrate.

7. The method according to claim 3, characterized by, if the measured concentration of the heavy metal ions in the sampled reactants is low in the step S7, then adding the plant for adsorbing heavy metal ions put in the waste water containing heavy metal ions.

8. The method according to claim 1, characterized by, further comprising:
In the late stage of methanation, the stable rubbish having been treated by the heavy metal ion enriched biomass waste material is landfilled at a final landfill; and the leachate is delivered to a urban domestic sewage treatment plant for deep treatment before being discharged.
